(19) United States
(12) Patent Application Publication
(10) Pub. No.: US 2026/0356383 A1
(43) Pub. Date: Nov. 12, 2026

(54) SMART VEHICLE SYSTEMS AND CONTROL LOGIC FOR PERFORMING AUTOMOTIVE PSYCHOMOTOR VIGILANCE TESTING

(71) Applicant: GM GLOBAL TECHNOLOGY OPERATIONS LLC, Detroit, MI (US)

(72) Inventors: Armando A. Beltran Pacheco, Rochester Hills, MI (US); Nomar S. Gonzalez Santini, Lakeland, FL (US); Rami I. Debouk, Dearborn, MI (US); Azeem Sarwar, Rochester Hills, MI (US); Maureen E. August, Grosse Pointe Woods, MI (US); Akilesh Rajavenkatanarayanan, Clinton Township, MI (US); Arun Adiththan, Sterling Heights, MI (US)

(73) Assignee: GM GLOBAL TECHNOLOGY OPERATIONS LLC, Detroit, MI (US)

(21) Appl. No.: 19/202,024

(22) Filed: May 8, 2025

(51) Int. Cl. B60W 50/08; A61B 5/16; A61B 5/18; B60W 10/18; B60W 40/08; B60W 50/14; B60W 60/00

(52) U.S. Cl. CPC B60W 50/082; A61B 5/168; A61B 5/18; B60W 10/18; B60W 40/08; B60W 50/14; A61B 2503/22; B60W 2040/0872; B60W 2050/143; B60W 2050/146; B60W 60/00; B60W 2520/06; B60W 2540/22; B60W 2540/229; B60W 2710/18

(57) ABSTRACT

Presented are smart vehicle systems for actively monitoring driver vigilance, methods for making/using such systems, and vehicles equipped with such systems. A method of operating a motor vehicle includes a vehicle controller receiving real-time sensor data indicating the vehicle is travelling forward and, in response, effecting a psychomotor vigilance test (PVT) analysis of the vehicle driver using a display device and a user input device mounted inside the vehicle's passenger cabin. Using PVT data collected from the PVT analysis, the controller generates a PVT data distribution for the driver and compares the PVT data distribution to a baseline PVT probability distribution to detect if the driver is impaired, i.e., the driver is experiencing either degraded driver vigilance or increased driver impulsivity. Upon detecting an impaired driver, the controller responsively commands a resident vehicle subsystem to perform a remediating action to redress the degraded driver vigilance or increased driver impulsivity.

### INTRODUCTION

The present disclosure relates generally to vehicle occupant monitoring systems. More specifically, aspects of this disclosure relate to smart vehicle systems for assessing driver impairment and automating ameliorative actions to mitigate an impaired driver.

Current production motor vehicles, such as the modern-day automobile, may be originally equipped with a network of resident controllers, sensors, and communications devices that enable a variety of vehicle services, such as advanced driver assistance system (ADAS) operations and automated passenger protection services. For instance, some automobiles are equipped with a Driver Alertness Monitoring (DAM) system that uses in-cabin sensors to assesses the driver's vigilance and, if cognitive impairment is detected, generates a visual, audible, or haptic alert to warn the driver. Some available DAM systems employ a CCD camera that is packaged inside the rearview mirror or steering column to track the driver's eye movement and head position. If the driver's head and eye activity indicates that they are not paying attention to the road ahead, and a precarious situation is detected, the DAM system will warn the driver by flashing a warning on the instrument cluster, emitting warning sounds on the cabin speaker system, or activating haptic transducers inside the steering wheel or driver seat.

### SUMMARY

Presented herein are smart vehicle systems with attendant control logic for monitoring driver alertness to assess sustained attention and reaction time, methods for manufacturing and methods for operating such systems, and motor vehicles equipped with such systems. By way of example, and not limitation, a closed-loop feedback control system actively evaluates driver vigilance and cognitive state through Automotive Psychomotor Vigilance Testing (APVT) both prior to and during driving. Pre-driving vigilance testing may be conducted off-vehicle using a dedicated mobile application (app) operating on a wireless-enabled personal computing device to assess the driver's alertness before driving commences. Active driving vigilance testing, in comparison, may be conducted in-vehicle using a head-up display (HUD) system and steering wheel controls during vehicle operation to continually monitor vigilance. The APVT system may track real-time psychomotor speed, lapses in attention, premature responses, and reaction times, etc., to provide a comprehensive view of the driver's cognitive performance and alertness.

Disclosed APVT systems and methods enhance and adapt established Psychomotor Vigilance Test (PVT) techniques, such as those used in aerospace and occupational science, into automotive applications by leveraging modern in-cabin human-machine interface (HMI) hardware and empirical data analytics to continually monitor driver vigilance. Cognitive function testing may be directly integrated into select vehicle systems, including head-up displays and telematics connectivity to smart mobile devices, to assess driver vigilance both before and during driving. Algorithmic vigilance data analysis may use a Vigilance Data Distribution Method (VDDM) algorithm to process and analyze vigilance data to detect patterns of drowsiness and impulsive behavior in drivers. This empirical data analysis framework may be tailored to an individual driver through a driver-calibrated PVT probability distribution model. Unlike current driver monitoring systems, which may require complex and costly wearable electronics, infrared-enabled cameras, and bio-monitors, disclosed APVT architectures offer a cost-effective and standardized systems to evaluate driver vigilance. This makes disclosed APVT systems more accessible, scalable, and practical for wider adoption in the automotive industry.

Aspects of this disclosure are directed to driver monitoring systems and system control logic for actively evaluating driver vigilance and mitigating impaired driver situations. In an example, a method is presented for operating a motor vehicle with a passenger cabin that contains a display device (e.g., an instrument panel (IP) mounted HUD device) and a user input device (e.g., steering wheel input controls). This representative method includes, in any order and in any combination with any of the above and below disclosed options and features: receiving, e.g., via a resident or remote microprocessor, central controller, control module, programmable logic circuit (PLC), integrated circuit (IC) device, or network of processors/controllers/modules/circuits/devices (collectively “vehicle controller”) from a vehicle dynamics sensor (e.g., wheel speed sensor, crankshaft position (CKP) sensor, etc.) and/or a vehicle control module (e.g., PCM, TCM, BCM, etc.), real-time vehicle data verifying the subject vehicle is moving in a forward or reverse direction; provisioning, e.g., via the vehicle controller responsive to the subject vehicle travelling forward or backward, a PVT analysis of the vehicle driver using the in-cabin display device and user input device; generating, e.g., via the vehicle controller, a discrete data distribution of PVT data collected from the PVT analysis of the vehicle driver; comparing, e.g., via the vehicle controller, the driver's PVT data distribution to a memory-stored baseline PVT probability distribution to determine whether or not the driver is in an impaired state, wherein the driver may be designated as impaired if they are experiencing degraded vigilance and/or increased impulsivity; and commanding, e.g., via the vehicle controller responsive to detecting an impaired driver state, one or more resident vehicle subsystem of the subject vehicle to perform one or more vehicle-calibrated remediating actions designed to redress the degraded driver vigilance or increased driver impulsivity.

Aspects of this disclosure are also directed to computer-readable media (CRM) containing controller-executable instructions for actively evaluating driver vigilance and mitigating impaired driver situations. In an example, a non-transient CRM stores instructions that are executable by a vehicle controller of a motor vehicle having a passenger cabin with a display device and a user input device both mounted inside the passenger cabin. These CRM-stored instructions, when executed, cause the vehicle controller to perform operations that include: receiving, from a vehicle dynamics sensor of the motor vehicle, vehicle driving data indicating the motor vehicle is travelling in a forward or reverse direction; effecting, responsive to the motor vehicle travelling in the forward or reverse direction, a psychomotor vigilance test analysis of a driver of the motor vehicle using the display device and the user input device inside the passenger cabin; generating a PVT data distribution of PVT data collected from the PVT analysis of the driver of the motor vehicle; comparing the PVT data distribution to a baseline PVT probability distribution to detect the driver is in an impaired driver state, the impaired driver state including either of a degraded driver vigilance and an increased driver impulsivity; and commanding, responsive to detecting the impaired driver state, a resident vehicle subsystem of the motor vehicle to perform a remediating action configured to redress the degraded driver vigilance or the increased driver impulsivity.

Additional aspects of this disclosure are directed to motor vehicles equipped with driver alertness monitoring systems for evaluating driver vigilance and mitigating impaired driver situations. As used herein, the terms “vehicle” and “motor vehicle” may be used interchangeably and synonymously to reference any relevant vehicle platform, such as passenger vehicles, commercial vehicles, industrial vehicles, tracked vehicles, off-road and all-terrain vehicles, farm equipment, motorcycles, watercraft, aircraft, spacecraft, etc. In an example, a motor vehicle includes a vehicle body with a passenger cabin, multiple road wheels attached to the vehicle body (e.g., via wheel corner modules coupled to a unibody or body-on-frame chassis), and other standard original equipment. A prime mover, which may be in the nature of an electric traction motor and/or an internal combustion engine (ICE) assembly, is located inside the vehicle body and drives the road wheel(s) to propel the vehicle. A head-up display device and a set of steering-wheel-mounted user input controls are both packaged inside the passenger cabin, located forward of the driver seat.

Continuing with the discussion of the foregoing example, the vehicle is also equipped with a resident or remote vehicle controller that is programmed to communicate—wirelessly or by-wire—with a personal computing device of the vehicle driver to receive therefrom pre-driving PVT data that is collected prior to driving of the subject vehicle. The vehicle controller generates a baseline PVT probability distribution using the pre-driving PVT data received from the driver's personal computing device. The controller then communicates with one or more resident vehicle dynamics sensors and/or one or more resident vehicle control modules to receive therefrom real-time driving data that indicates the motor vehicle is driving in a forward direction. Upon confirming that the vehicle is in drive (D) and travelling forward, the controller may responsively perform a PVT analysis of the vehicle driver using the in-cabin HUD device and steering-wheel-mounted user input controls. The controller concomitantly generates a discrete data distribution chart of PVT data collected by the PVT analysis of the vehicle driver and compares this PVT data distribution to the baseline PVT probability distribution to determine whether or not the driver is in an impaired state (e.g., overly fatigued, distracted, etc.). The diver may be deemed to be “impaired” if the data distribution comparison indicates they are experiencing measurable degraded vigilance and/or increased impulsivity. Upon determining that the driver is in an impaired state, the controller may responsively command one or more of the vehicle's resident vehicle subsystems to perform one or more remediating actions each designed to redress the degraded driver vigilance and/or increased driver impulsivity.

For any of the disclosed vehicles, methods, and CRM, the vehicle controller may calculate a statistical skewness of the driver's PVT data distribution, which is then compared to a symmetrical shape of the baseline PVT probability distribution to predict whether or not the driver is in an impaired state. For instance, the controller may determine whether or not the statistical skewness (SS) is less than or equal to a predefined normal threshold (NT) (e.g., SS≤NT); if not (i.e., statistical skewness greater than normal threshold), the driver may be deemed impaired and the resident vehicle subsystem(s) may automatically perform the vehicle-calibrated remediating action(s) to ameliorate the detected driver impairment. Conversely, the driver may be deemed “unimpaired” when the statistical skewness is less than or equal to the predefined normal threshold; in this instance, the vehicle controller does not command the resident vehicle subsystem(s) to perform the vehicle-calibrated remediating action(s).

For any of the disclosed vehicles, methods, and CRM, the vehicle controller may respond to detecting driver impairment (e.g., SS>NT) by determining whether or not the statistical skewness is greater than a predefined maximum skewness (MS) threshold and, thus, the driver is experiencing severely degraded vigilance. If so (e.g., SS>MS), the resident vehicle subsystem(s) may responsively perform one or more critical Driver Vigilance Degradation (DVD) responses (e.g., disable some or all autonomous vehicle (AV) driving features and activate vehicle limp home mode). Upon determining that the driver is not experiencing severely degraded vigilance (e.g., SS<MS), the vehicle controller may responsively determine whether or not the statistical skewness is less than a predefined negative skewness (NS) threshold and, thus, the driver is experiencing severely increased impulsivity. If so (e.g., SS<NS), the resident vehicle subsystem(s) may responsively perform one or more moderate DVD responses (e.g., disable select AV driving features).

For any of the disclosed methods, CRM, and vehicles, the vehicle controller may calculate a mean value, a median value, and/or a mode value of the PVT data distribution, which is/are then compared to a peak (mean) value of the baseline PVT probability distribution to predict whether or not the driver is in an impaired state. For instance, upon determining that the driver is not experiencing severely degraded vigilance or severely increased impulsivity, the controller may responsively determine whether or not the mean value is greater than the median value and the median value is greater than the mode value and, thus, the driver is experiencing mildly increased impulsivity. If so (e.g., mean>median>mode), the resident vehicle subsystem(s) may responsively perform one or more mild DVD responses (e.g., output visual and audible alerts on digital IP cluster, centerstack telematics unit, and vehicle speaker system). If the driver is not experiencing increased impulsivity, the controller may responsively determine whether or not the mode value is greater than the median value and the median value is greater than the mean value and, thus, the driver is experiencing mildly degraded vigilance. If so (e.g., mode>median>mean), the resident vehicle subsystem(s) may responsively perform one or more mild DVD responses.

For any of the disclosed methods, CRM, and vehicles, the resident vehicle subsystem may include an ADAS module that controls autonomous driving of the subject vehicle. In this instance, the remediating action may include the ADAS module disabling activation of one or more select autonomous driving features. As a further option, the resident vehicle subsystem may include an in-cabin infotainment system with a centerstack telematics unit, a digital instrument cluster, and/or a network of audio speaker components. In this instance, the remediating action may include the infotainment system outputting a predefined audible and/or visual alert of the impaired driver state. Visual and audible alerts may be supplemented with, or replaced by, tactile cues output by a steering wheel or seat-mounted haptic transducer The resident vehicle subsystem may also include a brake system control module (BSCM) that is operable to control the vehicle's brake system and/or a Steering Control Module (SCM) that is operable to control the vehicle's steering system. In this instance, the remediating action may include the BSCM activating the vehicle brake system and/or the SCM controlling the vehicle steering system. As used herein, “effecting” a PVT analysis may include displaying a series of visual prompts on the in-cabin display device at random intervals, concurrently tracking manual responses entered by the driver through the in-cabin user input device while displaying the visual prompts, measuring reaction times for the driver's responses, and recording a total number of response lapses and a total number of premature responses in the driver responses.

The above summary does not represent every embodiment or every aspect of the present disclosure. Rather, the foregoing summary merely provides a synopsis of some of the novel concepts and features set forth herein. The above features and advantages, and other features and attendant advantages of this disclosure, will be readily apparent from the following Detailed Description of illustrated examples and representative modes for carrying out the disclosure when taken in connection with the accompanying drawings and appended claims. Moreover, this disclosure expressly includes any and all combinations and subcombinations of the elements and features presented above and below.

### BRIEF DESCRIPTION OF THE DRAWINGS

FIG. 1 is a partially schematic, side-view illustration of a representative motor vehicle with an in-cabin head-up display (HUD) device and steering-wheel-mounted user input controls along with a network of in-vehicle controllers, sensors, and communication devices for provisioning psychomotor vigilance testing in accord with aspects of the present disclosure.

FIG. 2 is a flowchart illustrating a representative smart vehicle control protocol for automotive psychomotor vigilance testing (APVT), which may correspond to non-transient, memory-stored instructions that are executable by a resident or remote processor, control module, programmable logic circuit, central controller, or other integrated circuit (IC) device or network of processors/controllers/modules/circuits/devices/etc., in accord with aspects of the present disclosure.

FIG. 3 is a partially schematic, perspective-view illustration of a representative vehicle passenger cabin with an APVT system for monitoring driver alertness to assess sustained attention and reaction time in accord with aspects of the present disclosure.

FIGS. 4A and 4B are graphs illustrating a representative baseline PVT probability distribution graph (FIG. 4A) and examples of discrete data distributions showing degraded driver vigilance and increased driver impulsivity (FIG. 4B) in accord with aspects of the present disclosure.

The present disclosure is amenable to various modifications and alternative forms, and some representative embodiments of the disclosure are shown by way of example in the drawings and will be described in detail herein. It should be understood, however, that the novel aspects of this disclosure are not limited to the particular forms illustrated in the above-enumerated drawings. Rather, this disclosure covers all modifications, equivalents, combinations, permutations, groupings, and alternatives falling within the scope of this disclosure as encompassed, for example, by the appended claims.

### DETAILED DESCRIPTION

This disclosure is susceptible of embodiment in many different forms. Representative embodiments of the disclosure are shown in the drawings and will herein be described in detail with the understanding that these embodiments are provided as an exemplification of the disclosed principles, not limitations of the broad aspects of the disclosure. To that extent, elements and limitations that are described, for example, in the Abstract, Introduction, Summary, Brief Description of the Drawings, and Detailed Description sections, but not explicitly set forth in the claims, should not be incorporated into the claims, singly or collectively, by implication, inference or otherwise. Moreover, recitation of “first”, “second”, “third”, etc., in the specification or claims is not per se used to establish a serial or numerical limitation; unless specifically stated otherwise, these designations may be used for ease of reference to similar features in the specification and drawings and to demarcate between similar elements in the claims.

For purposes of this disclosure, unless specifically disclaimed: the singular includes the plural and vice versa (e.g., indefinite articles “a” and “an” should generally be construed as meaning “one or more”); the words “and” and “or” shall be both conjunctive and disjunctive; the words “any” and “all” shall both mean “any and all”; and the words “including,” “containing,” “comprising,” “having,” and the like, shall each mean “including without limitation.” Moreover, words of approximation, such as “about,” “almost,” “substantially,” “generally,” “approximately,” and the like, may each be used herein to denote “at or nearly at” or “same or practically the same” or “within 0-5% of” or “within acceptable manufacturing tolerances,” or any logical combination thereof, for example. Lastly, directional adjectives and adverbs, such as fore, aft, inboard, outboard, starboard, port, vertical, horizontal, upward, downward, front, back, left, right, etc., may be with respect to a motor vehicle, such as a forward driving direction of a motor vehicle when the vehicle is operatively oriented on a horizontal driving surface.

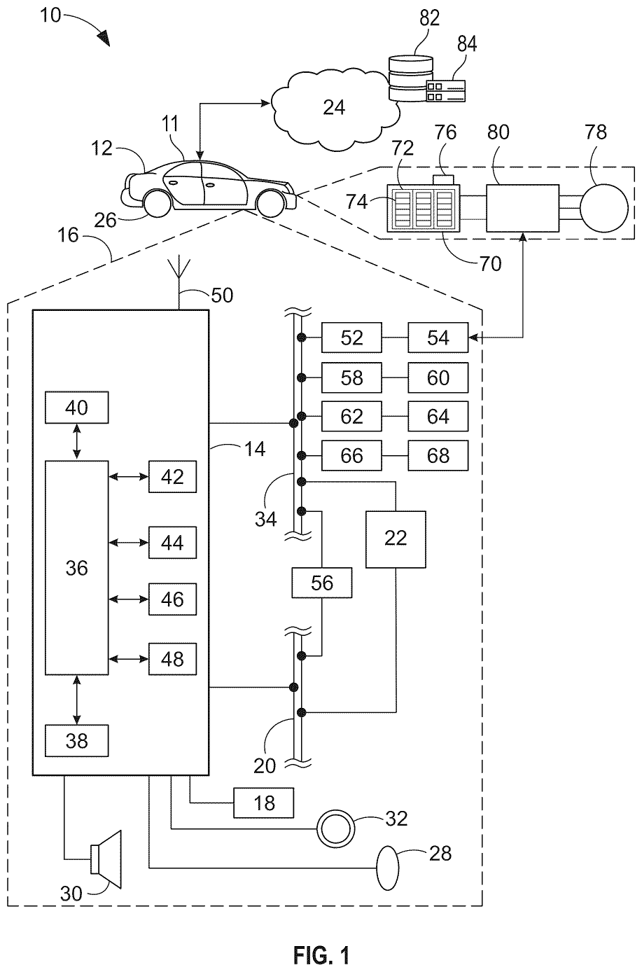

Referring now to the drawings, wherein like reference numbers refer to like features throughout the several views, there is shown in FIG. 1 a representative motor vehicle, which is designated generally at 10 and portrayed herein for purposes of discussion as a sedan-style, electric-drive automobile. The illustrated automobile 10—also referred to herein as “motor vehicle” or “vehicle” for short—is merely an exemplary application with which novel aspects of this disclosure may be practiced. In the same vein, incorporation of the present concepts into the specific Driver Monitoring System (DMS) architecture presented in the drawings should also be appreciated as a non-limiting implementation of disclosed features. As such, it will be understood that novel aspects of this disclosure may be implemented by other DMS architectures and may be incorporated into any logically relevant type of motor vehicle. Moreover, only select components of the motor vehicle and the DMS subsystem are shown and described in detail herein. Nevertheless, the vehicles and systems discussed below may include numerous additional and alternative features, and other available peripheral hardware, for carrying out the various methods and functions of this disclosure.

The representative vehicle 10 of FIG. 1 is originally equipped with a vehicle telecommunications and informatics (“telematics”) unit 14 that wirelessly communicates, e.g., via cellular network, satellite service, wireless-enabled modem, etc., with a remotely located cloud computing host service 24 (e.g., ONSTAR®). Some of the other vehicle hardware components 16 shown generally in FIG. 1 include, as non-limiting examples, an electronic video display device 18, a microphone 28, audio speaker(s) 30, and assorted user input controls 32 (e.g., buttons, knobs, dials, switches, touchpads, touchscreens, etc.). These hardware components 16 function, in part, as a human/machine interface (HMI) that enables a user to communicate with the telematics unit 14 and other components resident to and remote from the vehicle 10. Microphone 28, for instance, provides occupants with a means to input verbal commands; the vehicle 10 may be equipped with an embedded voice-processing unit with audio filtering, editing, and analysis modules. Conversely, the speaker 30 provides audible output to a vehicle occupant and may be either a stand-alone speaker dedicated to the telematics unit 14 or may be part of an in-cabin audio system 22. The audio system 22 is connected to a network connection interface 34 and an audio bus 20 to receive analog and digital signals, rendering it as sound, via one or more audio speaker components.

Communicatively coupled to the telematics unit 14 is a network connection interface 34, suitable examples of which include twisted pair/fiber optic Ethernet switches, parallel/serial communications buses, local area network (LAN) interfaces, controller area network (CAN) interfaces, and the like. The network connection interface 34 enables the vehicle hardware 16 to send and receive signals with one another and with various systems both onboard and off-board the vehicle body 12. This allows the vehicle 10 to perform assorted vehicle functions, such as modulating powertrain output, activating friction and regenerative brake systems, controlling vehicle steering, and other automated functions. For instance, telematics unit 14 may exchange signals with a Powertrain Control Module (PCM) 52, an Advanced Driver Assistance System (ADAS) module 54, a Brake System Control Module (BSCM) 56, a Body Control Module (BCM) 58, a Sensor System Interface Module (SSIM) 60, and assorted other vehicle ECUs, such as an Engine Control Module (ECM), a Motor Control Module (MCM), a Sensing and Diagnostics Module (SDM), a Transmission Control Module (TCM), a Steering Control Module (SCM), etc.

With continuing reference to FIG. 1, telematics unit 14 is an onboard computing device that provides a mixture of services, both individually and through its communication with other networked devices. This telematics unit 14 may be generally composed of one or more processors 40, each of which may be embodied as a discrete microprocessor, an application specific integrated circuit (ASIC), or a dedicated control module. Vehicle 10 may offer centralized vehicle control via a central processing unit (CPU) 36 that is operatively coupled to a real-time clock (RTC) 42 and one or more electronic memory devices 38, each of which may take on the form of a CD-ROM, magnetic disk, IC device, solid-state drive (SSD) memory, hard-disk drive (HDD) memory, phase-change memory (PCM), flash memory, semiconductor memory (e.g., various types of RAM or ROM), etc.

Long-range communication (LRC) capabilities with remote, off-board devices may be provided via one or more or all of a cellular chipset/component, a navigation and location chipset/component (e.g., global positioning system (GPS) transceiver), a wireless modem, or a mobile hotspot, all of which are collectively represented at 44. Close-range wireless connectivity may be provided via a short-range communication (SRC) device 46 (e.g., a BLUETOOTH® unit or near field communications (NFC) transceiver), a dedicated short-range communications (DSRC) component 48, and/or a dual RF antenna 50. The communications devices described above may provision data exchanges as part of a periodic broadcast in a vehicle-to-vehicle (V2V) communication system or a vehicle-to-everything (V2X) communication system, e.g., Vehicle-to-Infrastructure (V2I), Vehicle-to-Pedestrian (V2P), Vehicle-to-Device (V2D), Vehicle-to-Cloud (V2C), etc.

CPU 36 receives sensor data from one or more sensing devices that use, for example, photo detection, radar, laser, ultrasonic, optical, infrared, or other suitable technology, including short range communications technologies (e.g., DSRC) or Ultra-Wide Band (UWB) radio technologies, for executing a controller-automated (AV/ADAS) driving operation or a vehicle navigation service. In accord with the illustrated example, the automobile 10 may be equipped with one or more digital cameras 62, one or more range sensors 64, one or more vehicle speed sensors 66, one or more vehicle dynamics sensors 68, and any requisite filtering, classification, fusion, and analysis hardware and software for processing raw sensor data. The type, placement, number, and interoperability of the distributed array of in-vehicle sensors may be adapted, singly or collectively, to a given vehicle platform for achieving a desired level of automated vehicle operation.

To propel the automobile 10, a vehicle powertrain is operable to generate and deliver tractive torque to one or more of the vehicle's drive wheels 26. The powertrain is represented in FIG. 1 by an electric traction motor (M) 78 that is operatively connected to a rechargeable energy storage system (RESS), which may be in the nature of a chassis-mounted traction battery pack 70. The traction battery pack 70 is generally composed of one or more battery modules 72 each containing a cluster of battery cells 74, such as lithium-class, zinc-class, nickel-class, or organosilicon-class cells of the pouch, prismatic, or cylindrical type. One or more prime movers, such as traction motor/generator (M) units 78, draw electrical power from and, optionally, deliver electrical power to the battery pack 70. A power inverter module (PIM) 80 electrically connects the battery pack 70 to the motor(s) 78 and modulates the transfer of electrical current therebetween. The battery pack 70 may include an integrated electronics package, such as a wireless-enabled cell monitoring unit (CMU) 76, that enables on-module management, cell sensing, etc.

Presented below are smart vehicle systems with attendant control logic for assessing driver vigilance by utilizing a Psychomotor Vigilance Test (PVT) that is specifically adapted for automotive applications. For example, a subject “host” vehicle may conduct a pre-driving PVT through a dedicated mobile application (app) operating on the driver's personal smartphone or tablet computer to assess the driver's alertness (e.g., assesses how past conditions have affected driver). In tandem, the host vehicle may implement an intra-driving PVT that is performed in-vehicle using a head-up display (HUD) and steering wheel controls for real-time driver monitoring while the vehicle is in motion (e.g., assesses how current driving conditions affect driver performance). The PVT may be adapted to measure key cognitive metrics, such as reaction times, attention lapses, psychomotor speed, and/or impulsivity that may be caused by sleep deprivation, fatigue, medical conditions, distractions, substance use, etc. A PVT data processing module may employ an algorithmic framework to process vigilance data using a Vigilance Data Distribution Method (VDDM), which detects data patterns that are indicative of degraded vigilance and impulsive driving behavior based on reaction time distributions and skewness thresholds. The VDDM algorithm may categorize vigilance data to produce a Vigilance Index Value (VIV) that inversely correlates with drowsiness levels, thereby allowing for real-time assessment of driver alertness.

To actively assess cognitive impairment, the host vehicle's driver monitoring system may include a mobile application interface that performs pre-driving vigilance testing and provides cognitive state feedback to the driver before they commence driving. The driver monitoring system may also employ an in-car HUD device that works in conjunction with hand-operated driver input controls to conduct driving vigilance tests, derive real-time cognitive metrics, and deliver immediate feedback during driving. A data visualization and feedback (DVF) module may generate detailed graphical representations of PVT data, including histograms and scatter plots correlating delay times with reaction times. Utilizing active driver monitoring to systematically conduct psychomotor vigilance testing prior to and/or during driving may enable the dynamic adaptation of automated vehicle features based on the driver's real-time vigilance levels. The driver monitoring system may incorporate both mobile and in-vehicle hardware interfaces that are designed to seamlessly transition vigilance data between off-vehicle and on-vehicle environments.

Unlike conventional driver monitoring systems, which often require complex and expensive solutions with infrared-enabled, high-definition (HD) cameras, embedded bio-sensors (e.g., ECG, EEG), and wearable electronic devices, disclosed APVT protocols provide a simpler, scalable, and cost-effective approach to evaluating driver vigilance. Moreover, disclosed APVT systems and methods may adapt available PVT techniques to integrate standardized and reliable testing methods that are proven to accurately measure cognitive alertness and reaction time. Automotive PVT integration may allow for continual tracking and evaluation of driver alertness both before and during driving with measurable improvements to occupant and pedestrian protection. Real-time driver monitoring may provide immediate driver feedback and may be used to automate ADAS intervention when needed. Implementing disclosed VDDM algorithms may also facilitate more detailed analysis and categorization of PVT data, which may enable faster and more accurate detection of drowsiness and impulsive behaviors while facilitating targeted preventive measures. By providing a user-friendly interface for visualizing PVT data and presenting driver feedback, the APVT system may increase user engagement and help to ensure that drivers are aware of their cognitive states and can take corrective actions as needed.

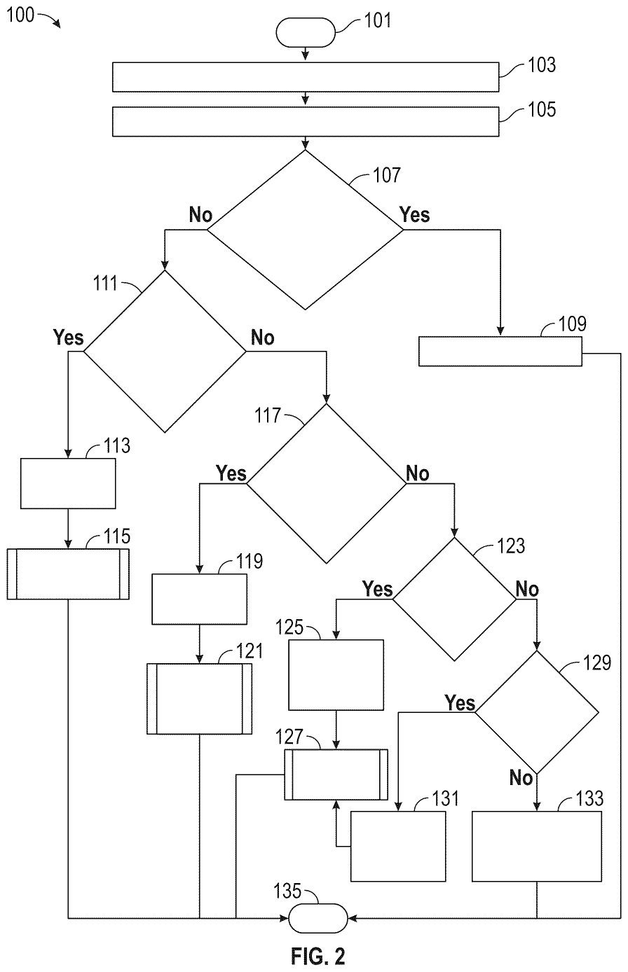

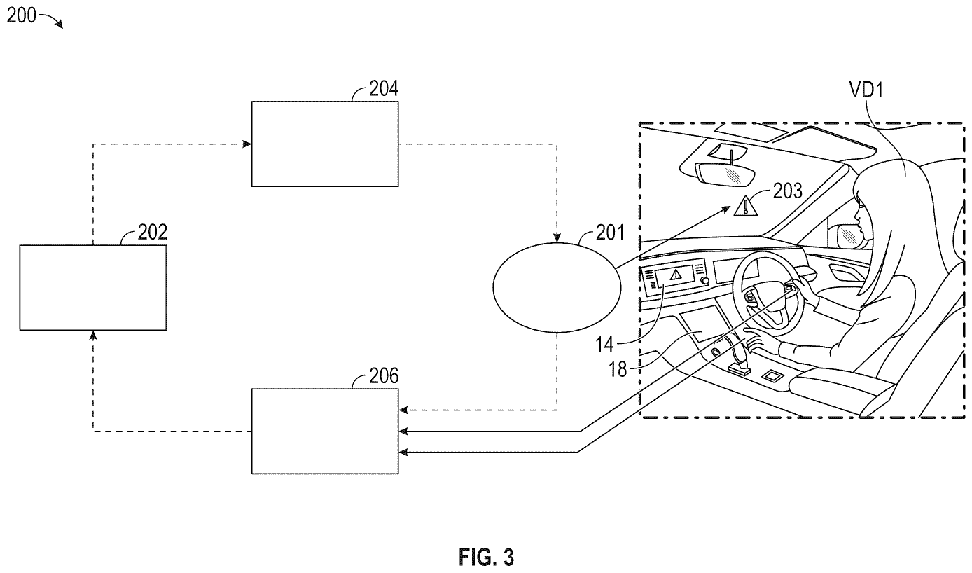

With reference next to the flowchart of FIG. 2, an improved method or control protocol for evaluating driver vigilance and remediating impaired driving of a vehicle, such as automobile 10 of FIG. 1, using a driver monitoring system, such as in-cabin DAM system 200 of FIG. 3, is generally shown at 100 in accordance with aspects of the present disclosure. Some or all of the operations illustrated in FIG. 2 and described in detail below may be representative of an algorithm that corresponds to non-transitory, processor-executable instructions that may be stored, for example, in main or auxiliary or remote memory (e.g., resident vehicle memory 38 and/or remote cloud service database 82 of FIG. 1). These instructions may be executed by a resident or remote microprocessor, central controller, control module, programmable logic circuit, integrated circuit (IC) device or other network of processors/controllers/modules/circuits/devices (e.g., vehicle CPU 36 and/or server-class cloud computing terminal 84 of FIG. 1) to perform any or all of the above and below described functions associated with the disclosed concepts. It should be recognized that the order of execution of the illustrated operation blocks may be changed, additional operation blocks may be added, and some of the herein described operations may be modified, combined, or eliminated.

Method 100 may begin at START terminal block 101 of FIG. 2 with memory-stored, computer-readable instructions for initializing a monitoring protocol for a driver of a motor vehicle. This routine may be initialized in real-time, near real-time, continuously, systematically, sporadically, and/or at predefined time intervals, for example, each 10 or 100 milliseconds during operation of the motor vehicle 10. As yet another option, terminal block 101 may initialize responsive to a user command prompt (e.g., via telematics input controls 32), a resident vehicle controller prompt (e.g., from CPU 36), or a broadcast prompt signal received from a centralized BO vehicle services system (e.g., from cloud host service 24). By way of non-limiting example, method 100 may automatically initialize during a key-on event in which a driver VD1 of the vehicle 10 powers on the vehicle powertrain, shifts the powertrain into a drive (D) mode, and commences vehicle launch. For pre-driving vigilance testing, method 100 may initialize in response to the driver VD1 opening a dedicated mobile app on their smartphone prior to entering the passenger cabin 11. Upon completion of some or all of the control operations presented in FIG. 2, method 100 may advance to END terminal block 135 and temporarily terminate or, optionally, may loop back to terminal block 101 and run in a continuous loop. Terminal block 135 may automatically trigger in response to a user shifting the powertrain into a neutral (N) or park (P) mode, or in response to a key-off event in which a user powers off the host vehicle.

After initialization at terminal block 101, method 100 may advance to PVT ANALYSIS data input block 103 and perform an Automotive Psychomotor Vigilance Test of the driver to aggregate a corresponding set of data vectors. By way of example, and not limitation, a resident APVT module 202 of DAM system 200 (FIG. 3) may communicate with the vehicle TCM or PCM 52 (FIG. 1) to confirm that the vehicle is in drive (D). APVT module 202 may concomitantly communicate through SSIM 60 with the resident vehicle speed or dynamics sensors 66, 68 to receive therefrom real-time driving data that verifies the vehicle 10 is presently driving in a forward direction. Method 100 may also verify that the host vehicle 10 has been driving for more than a preset threshold time (e.g., at least 30 minutes (mins)) and/or is driving faster than a preset threshold speed (e.g., above 25 miles per hour (mph)) before performing a PVT analysis. It is also envisioned that the driver may be prompted to approve vigilance testing prior to commencing a PVT analysis.

Upon confirming that the host vehicle 10 is in drive and travelling forward, APVT module 202 may responsively perform a PVT analysis 201 of the vehicle driver VD1 using a vehicle display device, such as in-cabin HUD device 204, in conjunction with hand-operated driver input controls, such as steering-wheel-mounted button set 206. Alternatively, the PVT analysis 201 may be carried out using other suitable HMI hardware, such as telematics unit 14, video display device 18, wireless-enabled personal computing device, and any of the assorted user input controls 32. The PVT analysis 201 may begin with the HUD device 204 displaying a series of visual prompts 203 at random intervals on the vehicle windshield, and concurrently tracking manual responses entered by the vehicle driver VD1 through the steering wheel button set 206. While displaying the visual prompts 203, the APVT module 202 measures the respective reaction times for each the driver's responses, and may simultaneously record a total number of response lapses (i.e., each time the driver VD1 failed to respond to prompt) and a total number of premature responses (i.e., each time the driver VD1 responded before a prompt was shown). In this manner, PVT may be implemented in a systematic way without adding hardware complexity to vehicles.

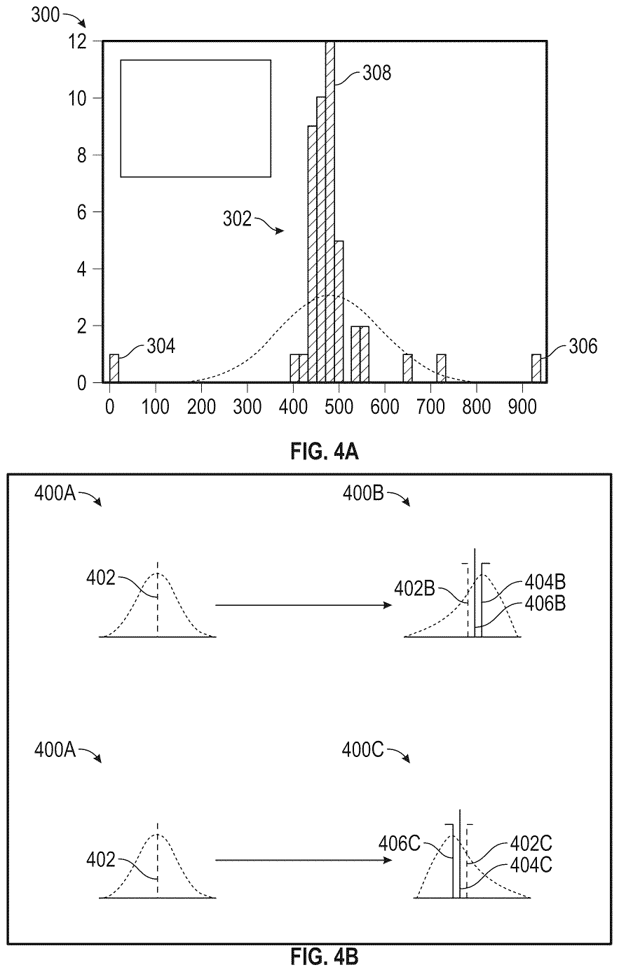

Turning back to FIG. 2, method 100 may proceed to PVT DATA DISTRIBUTION subroutine 105 and generate a discrete data distribution of the PVT data that is collected from the PVT analysis of the vehicle driver. FIG. 4A provides one example of a baseline PVT probability distribution 300 in which one or more historical PVT data sets are arranged into a histogram-type normal probability distribution of reaction times (in milliseconds (ms) on the horizontal X-axis) versus number of occurrences (on the vertical Y-axis). The baseline PVT probability distribution 300 is a 5-min standard PVT distribution constructed from histogram bars 302 that each corresponds to a count of data points falling within a respective range of reaction times. The left-most histogram bar 304 may represent premature responses (e.g., “false start” impulsivity induced by fatigue), whereas a right-most histogram bar 306 may represent failures to respond (e.g., response lapses indicating wake-state instability) and the central-most histogram bar 308 may represent a psychomotor speed (e.g., rate at which driver performs physical responses from mental reactions to visual stimuli). In this illustrated example, the mathematical mean is approximately 482 ms, the median is approximately 474 ms, the mode is approximately 449 ms, and the standard deviation is approximately 112.

Baseline probability distributions for PVT evaluations may be generated from historical or crowd-sourced data, as described above, or may be derived by conducting a pre-driving PVT analysis before the driver commences driving. For instance, FIG. 4B provides samples of three different PVT data sets that are arranged into respective discrete data distributions: (1) 400A is a normal (Gaussian) continuous probability distribution that represents a driver PVT baseline (e.g., generated during a pre-driving PVT); (2) 400B is a negatively skewed continuous probability distribution that implicates driver vigilance degradation (e.g., generated during an intra-driving PVT); and (3) 400C is a positively skewed continuous probability distribution that implicates driver impulsivity incrementation (e.g., generated during an intra-driving PVT). The normal continuous probability distribution 400A may have a substantially symmetrical “bell curve” shape in which the mathematical mean, median, and mode (collectively 402) are approximately the same and correspond to the center-most peak value. Comparatively, the negatively skewed continuous probability distribution 400B has a right-leaning shape in which the mean (center) 402B is skewed to the left of the median (middle value) 404B and the mode (most frequent value) 406B, whereas the positively skewed continuous probability distribution 400C has a left-leaning shape in which the mean 402B is skewed to the right of the median 404B and mode 406B.

A Vigilance Data Distribution Method (VDDM) may be employed for monitoring and analyzing driver performance. PVT DATA DISTRIBUTION subroutine 105 of FIG. 2, for example, may also include calculating a mean value, a median value, and/or a mode value for the PVT data distribution generated for each set of PVT data collected during a PVT analysis. As noted above, a mean value 402B, a median value 404B, and a mode value 406B may be calculated for the negative-skewed probability distribution 400B, and a mean value 402C, a median value 404C, and a mode value 406C may be calculated for the positive-skewed probability distribution 400C. The calculated mean, median, and mode values of the subject PVT data distribution may be compared to the peak value (mean/median/mode) of the baseline PVT probability distribution to assess driver impairment, as will be explained in detail below.

PVT DATA DISTRIBUTION subroutine 105 may also include calculating a statistical skewness of the PVT data distribution generated for each PVT data set. In statistics and probability theory, skewness may be defined as a statistical measure of the asymmetry of a probability distribution with respect to its mathematical mean. For nonparametric skew, skewness (γ) may be calculated as:

γ=(μ−υ)/σ

where μ is the mean, υ is the median, and σ is the standard deviation. The continuous probability distribution 400B of FIG. 4B may have a high negative skew with a skewness value of −1.3 (i.e., with a longer tail on the left side), and the continuous probability distribution 400C of FIG. 4B may have a high positive skew with a skewness value of 1.1 (i.e., with a longer tail on the right side). The statistical skewness of a PVT data distribution may be compared to the symmetrical shape of a baseline PVT probability distribution (e.g., with a skewness value of about zero (0)).

After generating the PVT data distribution for the PVT data collected from the PVT analysis, method 100 compares the PVT data distribution to a baseline PVT probability distribution to predict whether the driver is in an impaired driver state (i.e., reduced cognitive vigilance) or an unimpaired driver state (i.e., adequate cognitive vigilance). In FIG. 4B, for example, a comparison of the asymmetrical shape of the continuous probability distribution 400B to the symmetrical shape of the driver PVT baseline 400A suggests that the vehicle driver VD1 is experiencing degraded driver vigilance. Conversely, a comparison of the asymmetrical shape of the continuous probability distribution 400C to the symmetrical shape of the driver PVT baseline 400A suggests that the vehicle driver VD1 is experiencing increased driver impulsivity. As will be explained in further detail below, the type and severity of the driver's impairment may be ascertained by evaluating the skewness and calculated mathematical averages of the PVT data distribution.

With reference again to FIG. 2, method 100 may advance from subroutine 105 to NORMAL SKEW decision block 107 to predict the severity of the driver's impairment, if any, by assessing the degree of asymmetry of the PVT data probability distribution, if any. For instance, APVT module 202 may determine whether or not the statistical skewness (SS) for a given PVT data distribution is less than or equal to a predefined normal threshold (NT) (e.g., SS≤NT; NT=0.5) and, thus, the driver is not experiencing a measurable degree of impairment. Upon determining that the statistical skewness of the PVT data probability distribution is in fact less than or equal to the normal threshold (Block 107=YES), method 100 may responsively execute NORMAL DISTRIBUTION process block 109. At this juncture, APVT module 202 may set a flag in resident cache memory 38 that the subject PVT data probability distribution is “normal” and, as such, the vehicle driver VD1 is deemed to be in an unimpaired state. Method 100 may thereafter advance to terminal block 135 and temporarily terminate or may loop back to terminal block 101 and run in a continuous loop. For at least some applications, successfully completing a PVT analysis may be set as a system requirement for engaging specific vehicle features, such as level-two (L2) and level-three (L3) autonomous vehicle (AV) driving. In this regard, APVT may be used to enable switching between different levels of vehicle automation.

When a driver is deemed to be impaired, the host vehicle may automate operation of one or more resident vehicle subsystem(s) to automatically perform a single or multiple vehicle-calibrated remediating action(s) that are specifically designed to ameliorate the detected driver impairment. Upon determining that the statistical skewness of the PVT probability distribution exceeds the normal threshold (Block 107=NO), method 100 may respond by executing MAX POS SKEW decision block 111 to determine whether or not the probability distribution's statistical skewness is greater than a predefined maximum skewness (MS) threshold (e.g., SS>MS; MS=1.0). In this case, the driver is likely experiencing increased Driver Impulsivity Increment (DII). If the statistical skewness is greater than the maximum skewness threshold (Block 111=YES), method 100 may respond by executing POSITIVE SKEW process block 113 and setting a flag in resident cache memory 38 that the subject PVT data probability distribution is “positively skewed” and, as such, the vehicle driver VD1 is deemed to be experiencing a severely impaired state.

In tandem with process block 113, method 100 of FIG. 2 may responsively execute CRITICAL RESPONSE subroutine 115 and command a resident vehicle subsystem to execute a critical Driver Vigilance Degradation (DVD) response. In one example, APVT module 202 may communicate through network connection interface 34 with ADAS module 54 and PCM 52 to respectively disable all AV driving features and activate a vehicle limp home mode, which restricts vehicle speed and acceleration to preset maximum allowable values. It is envisioned that the host vehicle may take additional or alternative ameliorative measures to redress the degraded driver vigilance, such as activating ignition block, initializing level-four (L4) AV driving, or any of the other DVD responses described herein. The foregoing measures may be supplemented with a visual, audible, and/or haptic warning to the vehicle occupants.

In response to finding that the statistical skewness of the PVT data distribution is less than the max skewness threshold (Block 111=NO), method 100 may proceed to MAX NEG SKEW decision block 117 to determine whether or not the statistical skewness is less than a predefined negative skewness (NS) threshold (e.g., SS<NS; NS=−1.0). In such cases, the driver is likely experiencing severely degraded vigilance. If so (Block 117=YES), method 100 may respond by executing NEGATIVE SKEW process block 119 and setting a flag in resident cache memory 38 that the subject PVT data distribution is “negatively skewed” and, as such, the vehicle driver VD1 is deemed to be experiencing a severely impaired state. At the same time, method 100 may responsively execute CIRITICAL RESPONSE subroutine 121 and command a resident vehicle subsystem to execute a moderate DVD response to thereby ameliorate the driver's impaired state. In one example, APVT module 202 may communicate through network connection interface 34 with ADAS module 54 and telematics unit 14 to respectively disable select AV driving features (e.g., L2-AV driving and ACC) and display a visual alert to the driver (e.g., IMPAIRED DRIVING DETECTED—PULL OVER AND REST).

Upon determining that the driver is not experiencing severely degraded vigilance or severely increased impulsivity (Block 111=NO && Block 117=NO), method 100 may automatically carry out POSITIVE SKEW decision block 123 to determine whether or not the PVT data distribution's mean value is greater than the median value and the median value is greater than the mode value (mean>median>mode). In this case, the driver may be experiencing mildly increased impulsivity. If so (Block 123=YES), method 100 may respond by executing MILD POS SKEW decision block 125 and setting a flag in resident cache memory 38 that the subject PVT data probability distribution is “slightly positively skewed” and, as such, the driver VD1 is deemed to be experiencing a moderately impaired (impulsive) state. Method 100 may concurrently execute MILD RESPONSE subroutine 127 and command a resident vehicle subsystem to execute a mild DVD response to redress the increased impulsivity. APVT module 202 of FIG. 3, for example, may communicate through network connection interface 34 with the telematics unit 14, audio system 22, and BCM 58 (which controls driver seat operation) to respectively output visual, audible, and haptic alerts to the driver.

If the driver is not experiencing increased impulsivity (Block 123=NO), method 100 may proceed to NEGATIVE SKEW decision block 129 to determine whether or not the PVT data distribution's mode is greater than its median and its median value is greater than its mean (mode>median>mean). In such cases, the driver is likely experiencing mildly degraded vigilance. If so (Block 129=YES), method 100 may respond by executing MILD NEG SKEW process block 131 and setting a flag in resident cache memory 38 that the subject PVT data probability distribution is “slightly negatively skewed” and, as such, the driver VD1 is deemed to be experiencing a moderately impaired (fatigued/distracted) state. At the same time, method 100 may execute the MILD RESPONSE subroutine 127 and command one or more of the resident vehicle subsystems to execute one or more mild DVD response to redress the degraded vigilance. On the other hand, method 100 may conclude that the vehicle driver VD1 is experiencing neither increased impulsivity nor degraded vigilance (Block 123=NO && Block 129=NO); in respond, method 100 executes NORMAL SKEW process block 133 and sets a flag in resident cache memory 38 that the subject PVT data probability distribution is “approximately normal”. Method 100 may contemporaneously output a visual alert that the driver is not impaired (PVT NORMAL) and may thereafter temporarily terminate at terminal block 135.

Detecting a significant Driver Vigilance Degradation in a pre-driving PVT may be a strong indicator of driver impairment (e.g., substance use, under influence, sedative medication. etc.); in such cases, extreme driver restrictions may be reinforced by disabling vehicle start. On the other hand, detection of a moderate DVD during an intra-driving test may be an indicator of driver drowsiness, distraction, or fatigue; in such cases, a moderate mitigation mechanism may be triggered, including turning on the in-cabin audio system or rolling down a driver-side window. Detecting a significant DVD during an intra-driving test may be a strong indicator of serious driver drowsiness, distraction, or fatigue; in such cases, significant driver restrictions may be reinforced by disengaging active cruise control (ACC) and other vehicle-automated driving features that may require prompt driver intervention or may further exacerbate driver impairment.

When a moderate to significant DVD has been detected during an active driving test, indicating driver drowsiness, distraction, fatigue, etc., a Contextual ADAS Mode may be triggered. Existing ADAS modes are typically not “context aware” in that they operate in the same manner based on a set of preselected parameter settings (e.g., fixed headway distance for ACC). Upon detection of significant DVD, the host vehicle may dynamically adapt these parametric settings based on the context, may restrict a current ADAS mode, or may transition to a different ADAS mode based on driver's current state. On the other hand, if there is no detected DVD, the host vehicle's ADAS parameters may be left in or returned to default or driver-selected settings. For moderate to significant DVD, the host vehicle may generate advance warnings to the driver and, optionally, may prompt the driver to approve or disable the automated remediating actions. As a further option, driver vigilance data may be shared with a third party stake holder with the intention to either reinforce mitigation actions or prevent the driver from being involved in a risky situation. Third parties might include a relative or guardian (e.g., in the case of teen drivers and senior drivers), company stakeholders (e.g., in the case of fleet or service drivers) or first responders (e.g., in the case of substance abusers).

Aspects of this disclosure may be implemented, in some embodiments, through a computer-executable program of instructions, such as program modules, generally referred to as software applications or application programs executed by any of a controller or the controller variations described herein. Software may include, in non-limiting examples, routines, programs, objects, components, and data structures that perform particular tasks or implement particular data types. The software may form an interface to allow a computer to react according to a source of input. The software may also cooperate with other code segments to initiate a variety of tasks in response to data received in conjunction with the source of the received data. The software may be stored on any of a variety of memory media, such as CD-ROM, magnetic disk, and semiconductor memory (e.g., various types of RAM or ROM).

Moreover, aspects of the present disclosure may be practiced with a variety of computer-system and computer-network configurations, including multiprocessor systems, microprocessor-based or programmable-consumer electronics, minicomputers, mainframe computers, and the like. In addition, aspects of the present disclosure may be practiced in distributed-computing environments where tasks are performed by resident and remote-processing devices that are linked through a communications network. In a distributed-computing environment, program modules may be located in both local and remote computer-storage media including memory storage devices. Aspects of the present disclosure may therefore be implemented in connection with various hardware, software, or a combination thereof, in a computer system or other processing system.

Any of the methods described herein may include machine readable instructions for execution by: (a) a processor, (b) a controller, and/or (c) any other suitable processing device. Any algorithm, software, control logic, protocol, or method disclosed herein may be embodied as software stored on a tangible medium such as, for example, a flash memory, a solid-state drive (SSD) memory, a hard-disk drive (HDD) memory, a CD-ROM, a digital versatile disk (DVD), or other memory devices. The entire algorithm, control logic, protocol, or method, and/or parts thereof, may alternatively be executed by a device other than a controller and/or embodied in firmware or dedicated hardware in an available manner (e.g., implemented by an application specific integrated circuit (ASIC), a programmable logic device (PLD), a field programmable logic device (FPLD), discrete logic, etc.). Further, although specific algorithms may be described with reference to flowcharts and/or workflow diagrams depicted herein, many other methods for implementing the example machine-readable instructions may alternatively be used.

Aspects of the present disclosure have been described in detail with reference to the illustrated embodiments; those skilled in the art will recognize, however, that many modifications may be made thereto without departing from the scope of the present disclosure. The present disclosure is not limited to the precise construction and compositions disclosed herein; any and all modifications, changes, and variations apparent from the foregoing descriptions are within the scope of the disclosure as defined by the appended claims. Moreover, the present concepts expressly include any and all combinations and subcombinations of the preceding elements and features.

What is claimed:

1. A method of operating a motor vehicle with a vehicle body containing a passenger cabin, the method comprising:
receiving, via a vehicle controller from a vehicle sensor and/or a control module, vehicle driving data indicating the motor vehicle is travelling in a forward or reverse direction;
effecting, via the vehicle controller responsive to the motor vehicle travelling in the forward or reverse direction, a psychomotor vigilance test (PVT) analysis of a driver of the motor vehicle using a display device and a user input device both mounted inside the passenger cabin;
generating, via the vehicle controller, a PVT data distribution of PVT data collected from the PVT analysis of the driver of the motor vehicle;
comparing, via the vehicle controller, the PVT data distribution to a baseline PVT probability distribution to detect the driver is in an impaired driver state, the impaired driver state including either of a degraded driver vigilance and an increased driver impulsivity; and
commanding, via the vehicle controller responsive to detecting the impaired driver state, a resident vehicle subsystem of the motor vehicle to perform a vehicle-calibrated remediating action configured to redress the degraded driver vigilance or the increased driver impulsivity.

2. The method of claim 1, further comprising calculating a statistical skewness of the PVT data distribution, wherein predicting the impaired driver state further includes comparing the statistical skewness of the PVT data distribution to a symmetrical shape of the baseline PVT probability distribution.

3. The method of claim 2, wherein predicting the impaired driver state further includes determining if the statistical skewness is less than or equal to a predefined normal threshold, and wherein commanding the resident vehicle subsystem to perform the vehicle-calibrated remediating action is further in response to the statistical skewness being greater than the predefined normal threshold.

4. The method of claim 3, further comprising:
determining the driver is in an unimpaired driver state in response to the statistical skewness being less than or equal to the predefined normal threshold; and
not commanding the resident vehicle subsystem to perform the vehicle-calibrated remediating action in response to the driver not being in the impaired driver state.

5. The method of claim 3, further comprising determining, responsive to the statistical skewness being greater than the predefined normal threshold, if the statistical skewness is greater than a predefined skewness threshold, wherein the vehicle-calibrated remediating action is a critical Driver Vigilance Degradation (DVD) response performed by the resident vehicle subsystem in response to the statistical skewness being greater than the predefined skewness threshold.

6. The method of claim 5, further comprising determining, responsive to the statistical skewness not being greater than the predefined skewness threshold, if the statistical skewness is less than a predefined negative skewness threshold, wherein the vehicle-calibrated remediating action is the critical DVD response performed by the resident vehicle subsystem in response to the statistical skewness being less than the predefined negative skewness threshold.

7. The method of claim 1, further comprising calculating a mean value, a median value, and/or a mode value of the PVT data distribution, wherein detecting the impaired driver state further includes comparing the mean value, the median value, and/or the mode value of the PVT data distribution to a peak value of the baseline PVT probability distribution.

8. The method of claim 7, wherein predicting the impaired driver state further includes determining if the mean value is greater than the median value and the median value is greater than the mode value, wherein the vehicle-calibrated remediating action is a mild Driver Vigilance Degradation (DVD) response performed by the resident vehicle subsystem in response to the mean value being greater than the median value and the median value being greater than the mode value.

9. The method of claim 8, wherein predicting the impaired driver state further includes determining if the mode value is greater than the median value and the median value is greater than the mean value, wherein the vehicle-calibrated remediating action is the mild DVD response performed by the resident vehicle subsystem in response to the mode value being greater than the median value and the median value being greater than the mean value.

10. The method of claim 1, wherein the resident vehicle subsystem includes a brake system control module (BSCM) operable to control a vehicle brake system of the motor vehicle, and the remediating action includes the BSCM activating the vehicle brake system.

11. The method of claim 1, wherein the resident vehicle subsystem includes an advanced driver assistance system (ADAS) module operable to control autonomous driving of the motor vehicle, and wherein the remediating action includes the ADAS module disabling activation of an autonomous driving feature.

12. The method of claim 1, wherein the resident vehicle subsystem includes an infotainment system with a telematics unit, a digital instrument cluster, and/or multiple speaker components mounted inside the passenger cabin, and the remediating action includes the infotainment system outputting a predefined audible and/or visual alert of the impaired driver state.

13. The method of claim 1, wherein effecting the PVT analysis includes displaying a series of visual prompts on the display device at random intervals, tracking driver responses entered by the driver through the user input device while displaying the visual prompts, measuring a reaction time for each of the driver responses, and recording a number of response lapses and a number of premature responses of the driver responses.

14. A non-transient, computer-readable medium storing instructions executable by a vehicle controller of a motor vehicle having a passenger cabin with a display device and a user input device both mounted inside the passenger cabin, the instructions, when executed, causing the vehicle controller to perform operations comprising:
receiving, from a vehicle sensor and/or a control module of the motor vehicle, vehicle driving data indicating the motor vehicle is travelling in a forward or reverse direction;
effecting, responsive to the motor vehicle travelling in the forward or reverse direction, a psychomotor vigilance test (PVT) analysis of a driver of the motor vehicle using the display device and the user input device inside the passenger cabin;
generating a PVT data distribution of PVT data collected from the PVT analysis of the driver of the motor vehicle;
comparing the PVT data distribution to a baseline PVT probability distribution to detect the driver is in an impaired driver state, the impaired driver state including either of a degraded driver vigilance and an increased driver impulsivity; and
commanding, responsive to detecting the impaired driver state, a resident vehicle subsystem of the motor vehicle to perform a remediating action configured to redress the degraded driver vigilance or the increased driver impulsivity.

15. A motor vehicle, comprising:
a vehicle body including a passenger cabin with a head-up display (HUD) device and a set of steering-wheel-mounted user input controls both mounted inside the passenger cabin;
a plurality of road wheels attached to the vehicle body;
a prime mover attached to the vehicle body and configured to drive at least one of the road wheels to thereby propel the motor vehicle; and
a vehicle controller programmed to:
receive, from a personal computing device of a driver of the motor vehicle, pre-driving psychomotor vigilance test (PVT) data collected prior to movement of the motor vehicle;
generate a baseline PVT probability distribution using the pre-driving PVT data received from the personal computing device of the driver;
receive, from a vehicle sensor and/or a control module of the motor vehicle, vehicle driving data indicating the motor vehicle is driving in a forward direction;
responsive to the motor vehicle driving in the forward direction, effecting a PVT analysis of the driver of the motor vehicle using the HUD device and the steering-wheel-mounted user input controls;
generate a PVT data distribution for the driver of the motor vehicle using PVT data collected from the PVT analysis;
compare the PVT data distribution to the baseline PVT probability distribution to detect the driver is in an impaired driver state, the impaired driver state including either of a degraded driver vigilance and an increased driver impulsivity; and
responsive to detecting the impaired driver state, command a resident vehicle subsystem of the motor vehicle to perform a remediating action configured to redress the degraded driver vigilance or the increased driver impulsivity.

16. The motor vehicle of claim 15, wherein the vehicle controller is further programmed to calculate a statistical skewness of the PVT data distribution, wherein predicting the impaired driver state further includes comparing the statistical skewness of the PVT data distribution to a symmetrical shape of the baseline PVT probability distribution.

17. The motor vehicle of claim 16, wherein predicting the impaired driver state further includes determining if the statistical skewness is less than or equal to a predefined normal threshold, and wherein commanding the resident vehicle subsystem to perform the remediating action is further in response to the statistical skewness being greater than or equal to the predefined normal threshold.

18. The motor vehicle of claim 17, wherein the vehicle controller is further programmed to determine, responsive to the statistical skewness not being less than or equal to the predefined normal threshold, if the statistical skewness is greater than a predefined skewness threshold, wherein the remediating action is a critical Driver Vigilance Degradation (DVD) response performed by the resident vehicle subsystem in response to the statistical skewness being greater than the predefined skewness threshold.

19. The motor vehicle of claim 18, wherein the vehicle controller is further programmed to determine, responsive to the statistical skewness not being greater than the predefined skewness threshold, if the statistical skewness is less than a predefined negative skewness threshold, wherein the remediating action is the critical DVD response performed by the resident vehicle subsystem in response to the statistical skewness being less than the predefined negative skewness threshold.

20. The motor vehicle of claim 19, wherein the vehicle controller is further programmed to calculate a mean value, a median value, and/or a mode value of the PVT data distribution, wherein detecting the impaired driver state further includes comparing the mean value, the median value, and/or the mode value of the PVT data distribution to a peak value of the baseline PVT probability distribution.
